(19) United States
(12) Patent Application Publication
(10) Pub. No.: US 2018/0030507 A1
(43) Pub. Date: Feb. 1, 2018

(54) METHOD AND APPARATUS RELATING TO TREATMENT OF A BLOOD SAMPLE FOR SEQUENCING OF CIRCULATING TUMOUR CELLS

(71) Applicant: PROKYMA TECHNOLOGIES LIMITED, Liverpool (GB)

(72) Inventors: Damian BOND, Southport (GB); Christopher STANLEY, Cambridge (GB)

(21) Appl. No.: 15/550,512

(22) PCT Filed: Feb. 5, 2016
(86) PCT No.: PCT/GB2016/050335

§ 371 Date: Aug. 11, 2017

(30) Foreign Application Priority Data
Feb. 13, 2015 (GB) 1502374.0
Sep. 7, 2015 (GB) 1515754.8

(51) Int. Cl. C12Q 1/68

(52) U.S. Cl. CPC C12Q 1/6806; C12Q 2535/122; C12Q 1/6886

(57) ABSTRACT

A method of detecting mutations in a CTC genome that uses a negative selection step to remove a proportion of non-CTCs from a blood sample. The negative selection step is followed by extraction of the DNA from the remaining enriched CTCs and then by dilution of the DNA to a very low concentration and preparing and sequencing two or more replicates of the final dilution.

### TECHNICAL FIELD

This invention relates to a method for treating a blood sample in a way to enable effective genomic DNA sequencing of circulating tumour cells to be carried out.

Circulating tumour cells (CTCs) are known to be present in the blood of patients with various cancers. They are rare cells and can often be present only at 1 to 1,000 cells per ml of blood. They are greatly outnumbered by other cells present in blood and this complicates analysis of CTCs, either by cytochemical or molecular techniques.

### BACKGROUND ART

The exact role of circulating tumour cells (CTCs) in cancer is still not completely understood. They are thought to be the mechanism whereby the cancer spreads throughout the body. The current understanding is that a mutation occurs in a cell, which inactivates the mechanisms which control cell growth. There are likely to be multiple mutations with different effects such as those that lead to resistance to medications. The levels of CTCs increase as the tumour burden increases and this feature is used to monitor the effectiveness of treatment in, for example, breast cancer using the CellSearch instrument from Veridex. There is a requirement to extract, enrich and purify CTCs prior to analysis.

Capturing a small number of CTCs in, say, a 10 mL volume of whole blood, which will contain around 40 to 100 million leukocytes and up to 55 billion red cells, is a major challenge.

A key issue in all these CTC enrichment approaches is the problem of the inherent fragility of CTCs.

Positive enrichment techniques, including immunomagnetic separation (IMS) using cell specific antibody-coated beads, or the alternative physical entrapment approaches that rely on size differences between CTCs and blood cells such as leukocytes, erythrocytes and platelets have been used in the past to select and capture CTCs. IMS procedures generally involve the use of a CTC-specific antibody, such as an anti EpCam. This antigen is an epithelial cell surface marker that is thought to be present only in circulating cells derived from a tumour and is not present on the surface of blood cells.

The disadvantage of all these positive CTC selection techniques is that they rely on a feature of the cell which may not be present in all CTCs. For example not all CTCs may express EpCam so they will not be detected by an IMS technique. Also, not all CTCs may be of a larger diameter than leukocytes in the blood, particularly when the cell is in an early developmental stage and perhaps most susceptible to treatment. These smaller cells will not be retained by a physical entrapment technique intended to enrich much larger cells. Thus, with positive selection techniques there is a risk that some CTCs will not be present in the enriched fraction and this will lead to a false negative diagnostic test result.

In some cancers, e.g. pancreatic, there seems to be no, or very limited expression of EpCam. In these cancers it is thought that the CTCs change their phenotype and can lose epithelial characteristics and become mesenchymal in character; this may be the stage where the CTC spreads throughout the body. The known anti EpCam antibody capture mechanism therefore may not work when the cancer is at its most hazardous and metastasizing throughout the body. This issue of failure to detect such CTCs is overcome in the present invention.

Negative selection techniques i.e. the removal of cells other than the CTCs from a sample are now under active consideration as a way of reducing false negative results. Negative selection techniques rely on the efficient removal of all blood cells that could compromise the detection of CTCs present in a sample, leaving only non-interfering blood cells and CTCs remaining in suspension. A negative selection process is therefore not dependent on any specific feature of a CTC cell and therefore is more widely applicable as an enrichment technique. However, especially when using molecular analysis methods such as PCR or sequencing to analyse mutations in a CTC genome it is desirable for the negative selection process to be highly effective in reducing both specific wild type background (from non-mutated genes in other nucleated cells) and general PCR interference by excess DNA derived from other nucleated cells in blood by ensuring efficient removal of leukocytes.

U.S. Pat. No. 7,205,157 B (JURGENSEN ET AL) Apr. 4, 2007 proposes separating rare cells from a sample fluid either by positive or negative selection using an appropriate antibody-coated magnetic bead by centrifuging in a tube containing a harvesting float. In the negative selection process CTCs are gathered by pipetting from an intermediate layer in the harvester. Both processes described are inadequate to meet the requirement of enabling effective sequencing of circulating tumour cells. The centrifugal process may well destroy some cells of interest and given that the cells are rare this has a significant impact on the usability of the final sample. The magnetic beads used are typically between 4 to 5 μm in diameter, which the inventors have found associate non-specifically with red cells causing significant aggregation and clumping in the sample, with unintended capture of the cells of interest in the clumps. In the negative selection process described in U.S. Pat. No. 7,205,157B the band of CTC cells formed is not deep and it is very difficult to pipette the CTC cells alone without capturing a significant quantity of the buffy layer above. Positive selection as described in U.S. Pat. No. 7,205,157B has the same drawback as other positive selection processes. Yang et al (Biotechnology and Bioengineering 2009 Vol. 102, No. 2) describe the use of red cell lysis followed by CD45+ cell removal in a negative selection process. Yang et al state that the complete removal of red cells is needed for optimal CTC detection.

The introduction of next generation sequencing (NGS) techniques has allowed analysis of the genomes of CTCs at a much more detailed level and it is now recognised that detection of oncogene mutations can provide valuable information to the clinician when treating a cancer patient. Milbury et al (COLD-PCR Enrichment of Rare Cancer Mutations prior to Targeted Amplicon Resequencing. Clinical Chemistry 2012 58:3 580-589) working with lung adenocarcinoma and colorectal tumour tissue reported the use of COLD-PCR (coamplification at lower denaturation temperature PCR) to generate mutation-specific amplicons followed by NGS sequencing. Milbury et al report that sequencing errors when using conventional PCR mutation amplification approaches were in the 1%-2% range whilst their COLD-PCR technique was able to enrich mutations above the error-related noise enabling reliable identification of mutation abundances of approximately 0.04% i.e. a 50 times increase in signal to noise performance.

WO 2014/165762 A (SAMUELS ET AL) 9 Oct. 2014 discloses a method for analysis of biological material including DNA that involves a first step of removing non-CTCs from a blood sample using standard techniques including immunomagnetic separation followed by extracting the DNA and generating a large number of compartments using an undiluted sample, with the aim of achieving one genome per compartment or less. Each compartment, which is typically an aqueous droplet, is then analysed in one embodiment using the standard methods of droplet digital PCR for the presence of the mutation of interest. WO 2014/165762A also suggests that the compartments can be analysed by NGS as an alternative way of detecting a mutation but does not suggest how this can be achieved realistically when thousands or millions of droplets have been generated.

The inventors have found that the combination of enrichment of CTCs by negative selection and NGS is an optimal CTC mutation detection approach as it combines two complementary technologies: firstly negative selection that reduces or preferably eliminates the incidence of false negatives in a diagnostic test by avoiding the use of unreliable positive selection techniques and secondly DNA sequencing to provide extensive sequence data that increases the accuracy and reliability of the analysis compared to simpler detection techniques such as PCR.

The recently developed NGS methods are relatively simple, automated techniques that are capable of generating a substantial amount of mutational sequence information. As such they are well suited to use in a diagnostic assay to initially determine the mutation profile of a cancer.

However, although the combination of negative cell selection and NGS is in principle a powerful approach to CTC analysis it is clear that current NGS techniques will only provide reliable information if the target CTC genome is present in an enriched sample at a frequency higher than 1 CTC genome:100 normal genomes i.e. 1 CTC cell:50 nucleated non-CTC cells. This is because, as also reported by Milbury et al, the initial PCR step used to amplify the gene sequence of interest is inherently ‘noisy’ due to base misincorporation by Taq DNA polymerases and so a mutation present below a frequency of 1% in a DNA sample extracted from nucleated cells in blood would be undetectable.

### DISCLOSURE OF INVENTION

According to the present invention a method of detecting a mutation in a blood sample comprises:
treating the blood sample to remove a portion of normal nucleated non-CTC cells;
purifying DNA from the treated sample;
diluting the purified DNA;
separating the diluted DNA into two or more replicates;
sequencing the DNA present in each replicate; and
identifying a mutation in the sequence if 1% or more of the sequencing reads on the replicates show said mutation.

Preferably the threshold for identifying a mutation is one of 2% or more, 3% or more, 5% or more, or 10% or more reads on a replicate showing said mutation.

Preferably the concentration of DNA in the dilution is 100 genomes per microliter or less. In a theoretical example if the frequency of CTC genomes is 1% after initially treating the blood sample to remove a portion of normal nucleated non-CTC cells and the final volume is 10 μl and 10 equal replicates of 1 μl each are prepared then a CTC genome present in a particular replicate would effectively be enriched by a further 10× over the wild type genomes present. The optimal concentration of total genomes in the final dilution is determined by the number of replicates that can practically be made and sequenced; clearly if the concentration is significantly higher than 100 genomes per microlitre then there will be a dwindling probability of enrichment of CTC genomes if only a limited number of replicates i.e. 10 or less are used. In this example the number of replicates must be correspondingly increased to achieve a useful enrichment, potentially making the method uneconomic. In the theoretical example given above where the frequency of CTC genomes present is 1% or less after treating the blood sample to remove a portion of normal nucleated non-CTC cells, then using a much lower concentration in the final dilution i.e. 10 genomes per microliter or less significantly reduces the probability of a CTC genome being present in any replicate. Thus the method of the invention requires an optimal concentration of total genomes in the final dilution to be matched with a realistic number of replicates to be sequenced.

In the invention, preferably the separation of the final diluted sample is into 2, 5 or more preferably 10 replicates. Obviously as the number of replicates of the final dilution sample is increased the enrichment factor of any CTC genome present in any replicate is increased accordingly; with a practical limitation being the cost of sequencing all the replicates. However as the cost of sequencing falls in the future and as improved automation and microfluidic techniques are introduced it will therefore be cost effective to increase the number of replicates sequenced on a routine basis to >10. This approach differs from that described in in WO2014/165752A as it uses a limited number of replicates (compartments) where each replicate contains a mixture of non-CTC and CTC genomes instead of sequencing a large number of compartments each containing a single genome.

In one embodiment of the invention capturing and removing a portion of nucleated non-CTC cells from the blood sample is by binding to specific non-CTC cell antibody-coated beads and separating the beads with captured nucleated non-CTC cells from the remaining sample preferably magnetically or by gravity. Suitable dense magnetic beads designed to capture cells from whole blood for use in treating the blood sample to remove a portion of normal nucleated cells are described in WO 2013/121216 A (STANLEY ET AL) 22 Oct. 2013 (High efficiency cell capture).

Beads are functionalised with means to attach antibodies e.g. protein A or streptavidin with biotinylated antibodies. Antibodies with specificity against CD45 can be used in the method. CD45 is a type 1 transmembrane protein present on all differentiated haematopoietic cells except erythrocytes and platelets. This includes all the DNA-containing leukocytes that the inventors wish removed. Antibodies against this antigen are utilised and in some cases against CD3 and CD14 antigens preferably in combination to provide greater efficiency of total leukocyte capture and removal.

Preferably the beads are between 20-150 μm in diameter, and more preferably 50-100 μm in diameter; thus substantially larger than the bead sizes contemplated in U.S. Pat. No. 7,205,157. Likewise, ideally the beads may have a density >1.5 g/mL and preferably between 2 and 5 g/ml to facilitate mechanically-induced movement through the viscous whole blood sample and capture of suspended leukocytes.

According to a second embodiment of the invention capturing and removing a portion of normal nucleated non-CTC cells from the blood sample is by density gradient centrifugation.

In a third embodiment of the invention capturing and removing a portion of normal nucleated non-CTC cells from the blood sample is by density gradient centrifugation. Essentially the invention can be summarized as involving a negative selection step to remove a proportion of non-CTCs from a blood sample, followed by extraction of the DNA from the remaining non-CTCs and the enriched CTCs, followed by dilution of the extracted DNA to a very low concentration and sequencing a number of replicates of the final dilution. Thus the overall process comprises two different enrichment steps for CTC DNA acting in series, the first step ensures the removal of a proportion of the non-CTC DNA by separating intact cells whilst the second step aims to generate at least one replicate containing CTC DNA due to a Poisson distribution of genomes occurring during the preparation of the replicates. The advantage of the second enrichment step is that it compensates for incomplete removal of the non-CTCs in the first step thereby allowing reliable detection of mutations in a background of PCR induced base misincorporation.

The inventors consider that a mutant sequence reported in an NGS process is considered to be reliable if it is detected in >1% of the NGS reads in all replicates or in one or more replicates, preferably more than 2% or more preferably more than 3% or more preferably more than 5% of the sequencing reads; if however a mutant sequence is reported to be present in less than 1% of the NGS reads then this can be attributed to PCR base misincorporation error and hence is not an unequivocal result.

There are many advantages of this enrichment of CTCs by the method of negative selection followed by limiting dilution and sequencing of multiple replicates.

Firstly, it does not matter what phenotype the CTC is expressing. The DNA-containing cells remaining after negative selection are CTCs sufficiently enriched to be effectively sequenced in combination with the limiting dilution/multiple replicates method.

Secondly, the CTCs can be sequenced when they are most likely in the process of spreading throughout the body, leading to the development of metastases. They are likely to be most vulnerable to therapeutic intervention during this stage when circulating as single cells.

Thirdly, it is possible that there are far more CTCs present in blood than have been detected by capture via EpCam surface antigen or by size selection. This improved method permits the isolation and analysis of these currently undetectable cells.

Fourthly, this provides a screening tool with the potential to improve cancer survival rates. In most cases in countries with access to advanced medical care, the patient survives the resection and treatment of the primary tumour. It is the development of multiple secondary tumours resulting from metastasis throughout the body that results in mortality. By the time these are detected, the tumours have embedded in peripheral tissues or major organs. CTC levels at the time of primary treatment are thought to be high, thereby reducing the purity target required in terms of the absolute quantity of leukocytes that need to be removed. By identifying the mutation in that patient at such an early stage using sequencing techniques, lower cost and routine molecular tests can be used subsequently, such as qPCR, to monitor the patient post treatment and detect the re-appearance of CTCs from the tumour; allowing immediate treatment before the secondaries can implant and start to grow.

Fifthly, this is a tool to monitor patient response to therapy and guide selection of therapy. Cancers are not static and contain various different mutations across a number of sub-populations of cells. These can lead to resistance to certain treatments through mechanisms such as blocking of transfer of the toxic agent into the cell to kill it. As with bacterial antibiotic resistance, clinicians are seeing the resistance profile of cancers changing as disease develops. Presumably the treatments kill off susceptible populations of cancer cells, but resistant CTCs can then proliferate and grow into resistant forms of cancer. So a clinician has the information to help select the therapy most likely to work for an individual patient and then use regular screening on a blood test during a course of treatment to observe in real time that it is working and if CTC levels start to increase to find out if a resistant form of cancer is emerging and change therapy accordingly.

The inventors believe that use of a red cell lysis step prior to the non-CTC nucleated cell removal step, as used by Yang et al, is not advantageous as it could lead to loss of fragile CTCs in the harsh cell lysis conditions used. Indeed Yang et al report that their red cell lysis step, followed by centrifugation to concentrate the remaining mononuclear cells, leads to losses of 33% of the latter cells. Presumably, since epithelial or mesenchymal-like CTCs cells in blood could be more fragile than the blood mononuclear cells, the losses of CTCs could be substantially higher.

The inventors have found that red cells can be left intact prior to removal of the non-CTC nucleated cells and they are then lysed in a subsequent step when the DNA from the remaining nucleated cells is extracted and purified. Haemoglobin from the red cells released by this lysis step is then removed in the standard DNA purification techniques prior to sequencing the CTC genome with NGS.

In an alternative procedure a portion or substantially all of the red cells in a blood sample can be removed in a first step by the use, for example, of specific anti red cell antibody-coated beads, followed by removal of nucleated non-CTCs in a second step involving specific anti non-CTC antibody-coated beads. Each of these steps can be repeated one or more times to ensure efficient removal of red cells and/or non-CTC cells. An advantage of removing the red cells prior to binding nucleated non-CTC cells to antibody-coated beads is that the very large excess of red cells in a sample (>1000×) can be inhibitory to the binding of the more limited numbers of other cells present; presumably through nonspecific steric hindrance effects. A suitable antibody for removal of all red cells present would be one with specificity for a universal antigen such as CD235a (glycophorin A).

According to the present invention a still further method of analysing the mutational profile in a CTC genome is characterised in comprising removing a proportion or substantially all of the red cells prior to removing at least a portion of nucleated non-CTC cells from a blood sample; subsequently lysing the enriched CTC cells and purifying the DNA; carrying out a limiting dilution step to reach a level of less than 100 genomes per microliter, preferably less than 20 genomes per microlitre: separating the diluted sample into 2 or more, or preferably 10, or more than 10 replicates; sequencing specific regions of the CTC genome in each replicate; identifying a mutation in the sequence if more than 1% or preferably more than 2% or more preferably more than 3% of the sequencing reads in a replicate show said mutation at specific regions of the CTC genome.

Alternatives to cell-specific antibodies for immobilisation to the capture beads include nucleic acid-based aptamers or lectins or polymeric antibody mimics.

### EXAMPLES OF THE INVENTION

### Example 1

The following is an example for analytical purposes of whole blood spiked with cultured PANC1 cells, illustrating a negative enrichment protocol that also involves a red cell removal step.

20 μl of streptavidin-derivatised magnetic beads (50-100 μm diameter, GE Healthcare) were added to an uncoated polystyrene microwell. Then 10 μl of biotinylated anti CD235a mouse monoclonal antibody (Abcam, Cambridge, UK) and 10 μl phosphate buffered saline buffer pH 7.5 (PBS) were added and the plate was incubated on a plate shaker at 200 rpm for 30 min at room temperature. The beads were then washed 3 times with PBS using magnetic separation to recover the beads from the wash buffer. 25 μl of 1/10 whole blood in PBS was added to the washed anti 235a antibody-coated beads followed by 16,000 PANC1 cells per well, n=3. (PANC1 cells are a pancreatic tumour cell line and act as a CTC-simulant in the example). The antibody-coated beads and spiked whole blood sample were then incubated for 30 min without shaking to remove red cells. The red cell-depleted supernatant was then aspirated and added to another uncoated microwell and 20 μl of anti CD45-coated magnetic beads, prepared according to the method above, were added and the sample was incubated for a further 30 min without shaking to remove CD45+ cells i.e. the nucleated non-CTC cells. The nucleated non-CTC cell-depleted supernatant was then removed and the remaining cells in suspension were lysed and the released DNA purified using the Roche Magnapure system. A LightCycler (Roche) quantitative real time PCR system was used to assess both the level of normal nucleated non-CTC cells and the PANC1 cells remaining after the bead extraction steps. PCR primers directed against the normal KRAS gene were used to quantitate normal DNA whilst the DNA derived from the spiked PANC1 cells was measured using PCR primers directed against the KRAS aspartate mutation at codon 12.

In a further experiment the procedure above was repeated with the addition of a second red cell removal step prior to the CD45+ cell removal step.

The results are set out in Table 1 attached showing Ct values obtained in the LightCycler PCR instrument from DNA extracted from spiked whole blood and from samples after negative selection of PANC1 cells.

TABLE 1

| | KRAS normal | KRAS |
|---|---|---|
| | (nucleated | mutation |
| Sample | non-CTC cells) | (PANC 1 cells) |
| Spiked whole blood prior | 26.39 | 29.99 |
| to extraction | | |
| a) Negative selection: | 32.61 | 28.19 |
| one red cell (anti 235a | | |
| antibody) and one | | |
| nucleated non-CTC cell | | |
| (anti CD45 antibody | | |
| removal steps) | | |
| b) Negative selection: | 35.13 | 28.31 |
| two red cell (anti 235a | | |
| antibody) and one | | |
| nucleated non-CTC (anti | | |
| CD45 antibody) removal | | |
| steps | | |

The results show that the negative selection protocol significantly reduced the nucleated non-CTC content in the whole blood sample leaving the majority of the spiked PANC1 cells in suspension. The reduction in nucleated non-CTC cells observed in procedure b) in Table 1 was approximately 500 times without measurable loss of PANC1 cells.

### Example 2

The following is an example of the application of the method of the invention to a whole blood sample taken from a late stage metastatic pancreatic cancer patient. In this example there was no separate red cell removal step and the procedure involved a specific leukocyte depletion step followed by direct cell lysis, purification of DNA, dilution, preparation of multiple replicates and finally an NGS protocol.

Streptavidin-coated magnetic agarose beads of 50-100 μm diameter (GE Healthcare, Little Chalfont, UK) were coated with biotinylated anti-CD45 monoclonal antibody (Abcam, Cambridge, UK) according to the method in Example 1. Then, 0.5 ml of the anti-CD45 antibody-coated beads were added to 7.5 ml whole blood from a 7.0 year old female patient diagnosed with late stage metastatic pancreatic cancer; the sample was provided by the Royal Liverpool Hospital, Pancreatic Cancer Unit with appropriate ethics approval. The beads and whole blood sample were then mixed on an end-to-end rotating table for 30 min at room temperature. Then the beads were drawn to the side of the tube using a magnet and the leukocyte-depleted supernatant was aspirated from the tube. An aliquot of 0.5 ml of the leukocyte-depleted supernatant was then lysed, released DNA purified and concentrated according to standard methods in the Roche Magnapure system. This instrument delivers a sample of purified DNA in 0.1 ml buffer and this was then diluted to an approximate concentration of 10 genomes per μl based on a p53 wild type sequence-based qPCR analysis. Then 10×1 μl aliquots were taken from the diluted 10 genomes per μl stock solution to make 10 replicates and each was subjected to library preparation, bar coding, clonal amplification and p53 gene sequencing using the Ion Torrent NGS system (Life Technologies Inc., Carlsbad, USA)

This dilution to 10 genomes/10 replicates procedure (“10G/10”) is illustrated in Table 2 which shows the data from the whole blood sample that had been processed prior to the NGS analysis using the anti-CD45 coated beads.

TABLE 2

| Replicate | Base Pair | | | | | |
|---|---|---|---|---|---|---|
| Number | T | A | C | G | % Mutation | Total Reads |
| 1 | 2293 | 1126 | 4 | 0 | 32.90 | 3423 |
| 2 | 660 | 0 | 1 | 0 | 0 | 661 |
| 3 | 365 | 0 | 1 | 0 | 0 | 366 |
| 4 | 3764 | 82 | 2 | 0 | 2.88 | 2848 |
| 5 | 84 | 0 | 1 | 0 | 0 | 85 |
| 6 | 256 | 0 | 0 | 0 | 0 | 256 |
| 7 | 490 | 0 | 0 | 0 | 0 | 490 |
| 8 | 413 | 0 | 0 | 0 | 0 | 413 |
| 9 | 204 | 0 | 0 | 0 | 0 | 204 |
| 10 | 1832 | 1 | 2 | 0 | 0.05 | 1836 |
| Total | 9361 | 1209 | 12 | 0 | | |
| % | 88.46 | 11.42 | 0.11 | 0.00 | | |

The mutant sequence (T>A) is present in 11.4% of the total reads from all the 10×1 μl aliquots in the Ion Torrent instrument. Specifically in replicate 1 32.9% of the reads were the mutation sequence and in replicate 4 2.88% were the mutation sequence; both were well above the minimum 1% threshold specified for reliable mutation detection. This was therefore a reliable detection of the p. E271V mutation in the blood of this pancreatic cancer patient. p.E271V is a known missense mutation in p53, exon 8, leading to loss of functionality.

A control experiment using blood from the same patient processed with uncoated beads that did not have anti-CD45 antibody on the surface is shown in Table 3.

TABLE 3

| Replicate | Base Pair | | | | % | |
|---|---|---|---|---|---|---|
| Number | T | A | C | G | Mutation | Total Reads |
| 1 | 14785 | 2 | 21 | 0 | 0.01 | 14808 |
| 2 | 1948 | 1 | 1 | 0 | 0.05 | 1950 |
| 3 | 1087 | 0 | 2 | 0 | 0 | 1089 |
| 4 | 2579 | 0 | 4 | 0 | 0 | 2583 |
| 5 | 2104 | 0 | 1 | 0 | 0 | 2105 |
| 6 | 2719 | 3 | 1 | 0 | 0.11 | 2723 |
| 7 | 8981 | 2 | 8 | 0 | 0.02 | 8991 |
| 8 | 587 | 1 | 2 | 0 | 0.17 | 590 |
| 9 | 750 | 1 | 0 | 0 | 0.13 | 751 |
| 10 | 4028 | 0 | 1 | 0 | 0 | 4029 |
| Total | 39568 | 10 | 41 | 0 | | 39619 |
| % | 99.87 | 0.025 | 0.10 | 0.00 | | |

In this case the known mutation was not detected above the 1% threshold set for the PCR base misincorporation in any of the 10 replicates sequenced. This indicated that, in this non-leukocyte depleted sample the CTC mutant genome was not sufficiently enriched to allow reliable mutation calling by NGS.

Table 4 shows the results from a third blood sample from the same patient that had not been treated with beads i.e. no treatment of the whole blood had been carried out. In this second control experiment the known mutation was not detected above the 1% threshold in any of the 10 replicates sequenced.

TABLE 4

| Replicate | Base Pair | | | | | |
|---|---|---|---|---|---|---|
| Number | T | A | C | G | % Mutation | Total Reads |
| 1 | 3179 | 0 | 0 | 0 | 0 | 3180 |
| 2 | 4313 | 5 | 3 | 0 | 0.12 | 4323 |
| 3 | 5608 | 2 | 4 | 0 | 0.04 | 5617 |
| 4 | 3846 | 0 | 13 | 1 | 0 | 3864 |
| 5 | 352 | 0 | 2 | 0 | 0 | 359 |
| 6 | 2990 | 0 | 14 | 1 | 0 | 3011 |
| 7 | 2154 | 0 | 0 | 0 | 0 | 2161 |
| 8 | 1183 | 0 | 3 | 0 | 0 | 1194 |
| 9 | 1432 | 0 | 1 | 0 | 0 | 1492 |
| 10 | 18872 | 0 | 47 | 0 | .01 | 18931 |
| Total | 43979 | 9 | 87 | 2 | | 44077 |
| % | | 0.02 | 0.20 | 0.00 | | |

In this second control experiment the known mutation was not detected above the 1% threshold in any of the 10 replicates sequenced

In conclusion, the results show that the mutation p. E271V was detected by NGS in the blood of the late stage pancreatic cancer patient provided the large excess of wild type DNA present in the sample was removed by the two enrichment steps of leukocyte specific antibody-coated beads followed by the 10G/10 limiting dilution/multiple replicates step. This example also confirms that it was not a requirement to remove the red cells from the blood sample prior to cell lysis and DNA purification. Since this procedure involved a non-specific cell lysis, purification and concentration protocol the resulting DNA solution may well contain enriched genomes or DNA fragments from non-leukocyte cells (the putative CTCs) as well as circulating tumour DNA (ctDNA). In this example it is not possible to distinguish between these two different sources of mutant sequences.

### Example 4

In this example two commercially-available negative selection methods for CTC enrichment in whole blood were used. The first method “RosetteSep” (StemCell Technologies Inc.) uses a monoclonal antibody directed against cell surface antigens on human hematopoietic cells (CD45, CD66b) to crosslink, leukocytes to rosettes of red blood cells formed using an anti-glycophorin A antibody. These “immunorosettes” are then pelleted by centrifugation through a buoyant density medium such as Ficoll-Paque where the CTCs remain unrosetted above the Ficol-Paque layer and can be recovered by aspiration.

The second negative selection method is the OncoQuick device (Greiner Bio-One) which uses a more refined density gradient formed by centrifugation. CTCs remain in the upper layer of the gradient above a porous membrane whilst the heavier and denser leukocytes are pelleted at the bottom of the tube during centrifugation. CTCs can then be recovered by aspiration.

The RosetteSep method was used first as a negative selection enrichment procedure on whole blood from three cancer patients. The method of the invention was used, with dilution to 10 genomes per microlitre and sequencing of 10 replicates (“10G/10”), to identify a p53 mutation in the CTC fraction, the mutation was not seen in the immunorosette pellet which would not be expected to contain CTCs (see Table 5).

TABLE 5

| | | | Enriched | Pellet |
|---|---|---|---|---|
| Patient | Diagnosis | Timing | p53 | p53 |
| 6 | Metastatic gastric | 3 days post | p.E271G | X |
| | adenocarcinoma | resection | p.T211A | X |
| | (ovary and pancreas) | | p.N200S | X |
| | | | p.R175H | X |
| 7 | Recurrent locally | 6 months post | p.K164R | X |
| | advanced PDAC | initial resection, | | |
| | | 3 days post | | |
| | | palliative bypass | | |
| 8 | Locally advanced PNET | 6 months post | p.E271G | X |
| | | palliative bypass | | |

Subsequently a combination of frozen and fresh whole blood samples was processed using both Oncoquik and RosetteSep. In this example samples were diluted to 20 genomes per microlitre and 2 replicates were sequenced (“20G/2”). No mutations were found in the 2 healthy controls. Mutations were identified in 3/7 patients with pancreatic cancer. The mutation in KRAS p.V14L was found in both the Oncoquik and RosetteSep-enriched samples in a fresh whole blood samples.

### Apparatus

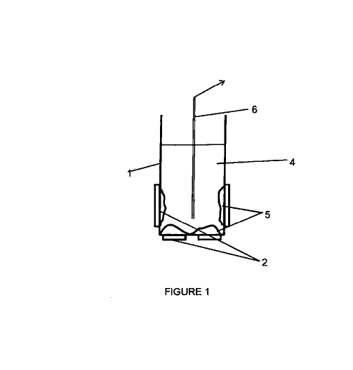

In FIG. 1 apparatus to negatively select CTC cells comprises a microwell 1 having magnets 2 (normally permanent magnets although electro-magnets can be used) attached to the bottom and possibly its sides. Streptavidin-derivatised magnetic beads 50-100 μm in diameter, and typically 3.5 g/ml in density (GE Healthcare) and coated with biotinylated anti CD45 antibodies were added to a blood sample containing suspected CTC cells, mixed and allowed to incubate for 30 minutes. The blood and antibody coated bead mixture is placed in the microwell 1. It was found that the large comparatively heavy beads captured the leukocytes in the blood efficiently without the clumping or aggregation of red cells seen with smaller lighter beads.

The blood and bead mix 4 is placed in the microwell; the beads 5 bind leukocytes and are attracted to the magnets 2 and congregate on the surfaces of the microcell 1 adjacent to the magnets 2; some also fall under gravity to the bottom of the microwell and are held by the magnets there.

The sample, now depleted in leukocytes, can be drawn from the microcell using a pipette 6, extending near to the base of the microcell, but remaining sufficiently clear of the bottom not to draw in beads and leukocytes that have been immobilised there.

The CTC-enriched sample remaining is now lysed and the released DNA prepared, preferably using the Roche Magnapure system.

For most CTCs, it will be found that the enriched blood sample prior to lysing will contain CTCs present at a frequency of 1:50 or higher.

Red blood cells could be removed from the sample after extracting leukocytes by extracting them in the same way using beads coated with biotinylated anti CD235a antibodies in the same way as described above. But as red cells contain no DNA to interfere with the subsequent steps there appears little advantage in doing this and possibly some risk in losing target CTCs in the process.

For some CTCs, particularly those derived from pancreatic cancer, it may be necessary to repeat the initial leukocyte separation prior to the lysing step in order to reduce further the numbers of any remaining leukocytes in the sample.

It will also be beneficial to coat the beads with anti CD3, anti CD14 and anti CD19 antibodies well as anti CD45 antibodies to improve the efficiency of removal of T-cells, monocytes, and B-cells. Further antibodies for other leukocyte cell surface antigens may be used.

1. A method of detecting a mutation in a blood sample that comprises: treating the blood sample to remove a portion of normal non-CTC nucleated cells; purifying DNA from the treated sample; diluting the purified DNA; separating the diluted DNA into two or more replicates wherein each replicate contains both CTC and non-CTC derived genomes; sequencing the DNA present in each replicate and identifying a mutation in the sequence if more than 1% of the sequencing reads on one or more replicates show said mutation.

2. A method according to claim 1 wherein the threshold for identifying a mutation is one of 2% or more, 3% or more, 5% or more, or 10% or more reads on a replicate showing said mutation.

3. A method according to claim 1 wherein the DNA is diluted to a level of 100 genomes per microliter or less.

4. A method according to claim 1 wherein the DNA is diluted to a level of 20 genomes per microliter or less.

5. A method according to claim 4 wherein the DNA is diluted to a level of 10 genomes per microliter or less.

6. A method according to claim 1 wherein the diluted DNA is separated into five or more replicates and each replicate is sequenced.

7. A method according to claim 6 wherein the diluted DNA is separated into 10 or more replicates and each replicate is sequenced.

8. A method according to claim 1 wherein a portion of non-CTC nucleated cells is removed using non-CTC cell antibody-coated beads.

9. A method according to claim 8 where the beads are coated with anti-CD45 antibody.

10. A method according to claim 9 where the beads are additionally coated with one or more of anti-CD45 antibody, anti-CD14 antibody, anti-CD19 antibody, and anti-CD3 antibody.

11. A method according to claim 8 wherein the beads are 20 to 150 μm inclusive in diameter.

12. A method according to claim 8 wherein the beads have a density of 1.5 g/mL or more.

13. A method according to claim 1 wherein a portion of non-CTC nucleated cells is removed using density gradient centrifugation.

14. A method according to claim 1 wherein a portion of non-CTC nucleated cells is removed by forming rosettes from red cells and binding non-CTC cells to said rosettes using non-CTC cell specific antibodies.

15. A method according to claim 1 wherein removal of a portion of non-CTC nucleated cells is preceded by removal of a portion or substantially all of the red cells present in the sample.
